(19) United States
(12) Patent Application Publication
(10) Pub. No.: US 2008/0008884 A1
(43) Pub. Date: Jan. 10, 2008

(54) Organic electroluminescent device and production method thereof, and impurity content determination method for aliphatic ketone solvent for use in preparation of organic electroluminescent device

(75) Inventors: Toru Ishii, Kanagawa (JP); Daisuke Okuda, Kanagawa (JP); Yohei Nishino, Kanagawa (JP); Akira Imai, Kanagawa (JP); Tadayoshi Ozaki, Kanagawa (JP); Hidekazu Hirose, Kanagawa (JP); Mieko Seki, Kanagawa (JP); Koji Horiba, Kanagawa (JP); Takeshi Agata, Kanagawa (JP); Kiyokazu Mashimo, Kanagawa (JP); Katsuhiro Sato, Kanagawa (JP); Hirohito Yoneyama, Kanagawa (JP)

Correspondence Address: OLIFF & BERRIDGE, PLC, P.O. BOX 320850, ALEXANDRIA, VA, 22320-4850, US

(73) Assignee: Fuji-Xerox Co., Ltd.

(21) Appl. No.: 11/703,457

(22) Filed: Feb. 7, 2007

(30) Foreign Application Priority Data
Jul. 5, 2006 (JP) 2006-185971

(51) Int. Cl. B32B 9/04; G01N 33/00

(52) U.S. Cl. 428/411.1; 73/61.43

(57) ABSTRACT

A method of producing an organic electroluminescent device includes forming an organic compound layer with an organic compound layer coating liquid that contains an organic compound and an aliphatic ketone solvent. The aliphatic ketone solvent contains about 0.01% by weight or less of an impurity contained ketone structure component.

### CROSS-REFERENCE TO RELATED APPLICATIONS

This application is based on and claims priority under 35 USC 119 from Japanese Patent Application No. 2006-185971 filed Jul. 5, 2006.

### BACKGROUND

1. Technical Field

The invention relates to a method for producing an organic electroluminescent device and an organic electroluminescent device produced thereby. Furthermore, the invention relates to an impurity content determination method for an aliphatic ketone solvent for use in preparation of an organic electroluminescent device.

2. Related Art

Electroluminescent devices (hereinafter, referred to as an “EL device”) are self-luminescent all solid-state devices with high visibility and impact resistance and, accordingly, a wide range of use applications are expected therefor. At present, EL devices that use an inorganic phosphor are main stream; however, there are problems in that since an AC voltage of 200 V or more is necessary for driving, production costs are high and the brightness is insufficient.

On the other hand, research on an EL device using an organic compound started with a single crystal of anthracene or the like. However, in the case of a single crystal, since the film thickness is as thick as about 1 mm, a driving voltage of 100 V or more was necessary. Accordingly, there have been attempts to obtain a thinner film by use of the vapor deposition method. However, a thin film obtained by this method still needs a high driving voltage of 30 V, and since electron/hole carrier density in the film is low and the generation probability of photons due to the recombination of carriers is low, sufficient brightness is not obtained.

However, in recent years, a separated-function type EL device has been reported, where a hole transporting organic low molecular weight compound and a fluorescent organic low molecular weight compound having electron transportability are sequentially layered by means of a vacuum vapor deposition method, which can obtain brightness as high as 1000 cd/m² or more at a low driving voltage of about 10 V. Since then, research and development of laminate type EL devices has been actively conducted. In a laminate type EL device, holes and electrons are injected from an electrode through a charge transport layer made of a charge transport organic compound, while maintaining a carrier balance between holes and electrons, into a luminescent layer made of a fluorescent organic compound, and the holes and electrons confined in the luminescent layer recombine to realize high brightness luminescence.

However, in the above EL devices, there are three major problems for putting into practical use as described in the following.

(1) Since a device is driven at such a high current density as several mA/cm², a large amount of Joule heat is generated. Accordingly, the hole transport low molecular weight compound or the fluorescent organic low molecular weight compound layered in an amorphous state by the vapor deposition method is, in many cases, gradually crystallized and finally melted to deteriorate the brightness or cause the dielectric breakdown. As the result, the lifetime of the element is deteriorated.

(2) When a device is prepared, since the low molecular weight organic compounds are formed in a thin film having a film thickness of 0.1 μm or less in a plurality of vapor deposition processes, pinholes tend to be generated. Accordingly, in order to obtain sufficient performance, a film thickness control under severely controlled conditions is necessary. As a result, the productivity is low and it is difficult to obtain larger area elements.

(3) What is utilized in a luminescent material is luminescence from an excited singlet state, that is, fluorescence. However, since a generation ratio of excitons of excited singlet state and excitons of excited triplet state generated when the holes and electrons recombine in the luminescent layer is 1:3 from the spin-statistics theorem based on quantum physics, the internal quantum efficiency of the luminescence in an organic EL device is theoretically 25% at most.

In order to overcome the problem shown in the (1), an EL element where star-burst amine with which as a hole transport material a stable amorphous glass state can be obtained is used and an EL element where a polymer in which, in a side chain of polyphosphazen, triphenylamine is introduced is used are reported (for instance, non-patent documents 3 and 4).

However, since in these devices alone there exists an energetic barrier caused by ionic potential of hole transport materials, these devices can not satisfy hole injection ability from an anode electrode or hole injection ability into luminescent layers. In the case of the former star-burst amine, it is difficult to clean up the amine since the amine is not suitable for purification due to low solubility, and in the case of the latter polymer there is a problem in that enough luminance is not available due to a lack of high current density.

Although, for the purpose of solving the problem described in the (2) above, research and development has been conducted for obtaining the EL devices having a single layer structure which shorten the manufacturing time and it has been proposed to use electrically conductive polymer such as poly(p-phenylene vinylene) or to mix electron transport material and luminescent dye into electron transport poly(vinyl carbazole), the properties such as luminance or luminescent efficiency of these compounds still hasn't come up to that of organic low molecular weight compounds which are used in EL devices with multilayer structures.

Further, regarding manufacturing methods wet coating process is preferable in the viewpoint of simplification, workability, large-area extendibility and cost. It is reported that the devices are formed by casting method. However, since there is a problem in that the electron transport materials easily crystallize due to low solubility and low compatibility to solvents or resins, the EL devices in which electron transport layers are formed by coating of electron transport polymer has been proposed.

However, since it is high possibility that coating solvents are released into air, appropriate countermeasures to prevent environmental pollution are necessary. Especially, avoiding the use of halogen solvents such as chlorofluorocarbons is preferable. Recently an aliphatic ketone solvent draw attention as a solvent having adequate boiling point and viscosity and having high dissolving power, and specifically cycloheptane which has superior solubility is useful as the non-halogen solvent having boiling point suitable for coating.

### SUMMARY

According to an aspect of the present invention, a method for producing an organic electroluminescent device includes forming an organic compound layer with an organic compound layer coating liquid that contains an organic compound and an aliphatic ketone solvent, the aliphatic ketone solvent containing 0.01% by weight or less of an impurity contained ketone structure component.

### DESCRIPTION OF THE DRAWINGS

Exemplary embodiments of the present invention will be described in detail based on the following Figures, wherein:

FIG. 1 is a schematic sectional view showing an example of an organic EL device of the invention;

FIG. 2 is a schematic sectional view showing another example of an organic EL device of the invention;

FIG. 3 is a schematic sectional view showing still another example of an organic EL device of the invention;

FIG. 4 is a schematic sectional view showing another example of an organic EL device of the invention;

FIG. 5 is a diagram showing relationship between purities and brightness of solvents obtained in examples and comparative examples;

FIG. 6 is a diagram showing relationship between purities and brightness-current efficiencies of solvents obtained in examples and comparative examples;

FIG. 7 is a diagram showing relationship between total concentrations (contents) of impurities and brightness of solvents obtained in examples and comparative examples; and

FIG. 8 is a diagram showing relationship between total concentrations (contents) of impurities and brightness-current efficiencies of solvents obtained in examples and comparative examples.

### DETAILED DESCRIPTION

After a hard research on trace components in aliphatic ketone solvents, it is discovered that, a content of trace particular components, rather than the purity itself of a solvent lot, affects largely on the luminescence characteristics of an organic electroluminescent device and thereby the invention came to completion.

In what follows, a method for producing an organic electroluminescent device according to an exemplary embodiment of the invention will be described in detail.

A method for producing an organic electroluminescent device in the exemplary embodiment includes forming an organic compound layer with an organic compound layer coating liquid that contains an organic compound and an aliphatic ketone solvent, wherein the aliphatic ketone solvent contains 0.01% by weight or less of an impurity contained ketone structure component.

In the method of producing an organic electroluminescent device in the exemplary embodiment, when, for instance, a charge transport layer (hole transport layer, electron transport layer), a luminescent layer or a luminescent layer having the carrier transportability is formed from an organic compound (for instance, polymer), a organic compound layer coating liquid in which the organic compound is dissolved (or dispersed) in a solvent is coated to form an organic compound layer.

As a solvent of the organic compound layer coating liquid, an aliphatic ketone solvent of which a content of impurity contained ketone structure is a predetermined value or less is used. When such an aliphatic ketone solvent is used, a device excellent in the brightness and the brightness-luminescence efficiency can be assuredly obtained.

Here, though why the impurity contained ketone structure in the aliphatic ketone solvent largely affects on the performance of the device is not clear, it is considered that even slight presence of a ketone group having a large dipole moment in an organic compound layer largely adversely affects on the carrier transportability.

In particular, in an aliphatic ketone solvent, an impurity contained ketone structure only slightly different in the number of carbon atoms from that of a main component tends to be generated as impurity and the physical properties of the substance are similar to the main component. Accordingly, it is assumed that removal efficiency in a refining process is much fluctuated due to external disturbances such as room temperature and pressure at the refining, and thereby quality of the solvent as a product is difficult to be constant. Furthermore, the impurity contained ketone structure slightly larger in the number of carbon atoms has a larger molecular weight and a relatively higher boiling point; accordingly, a ketone group having a large dipole moment that is expected to largely affect on the transportation performance of the carriers is considered likely to remain in the organic layer.

Such an organic compound layer coating liquid includes at least an organic compound corresponding to an object and an aliphatic ketone solvent as a solvent.

The aliphatic ketone solvent will be described. In the aliphatic ketone solvent, an impurity contained ketone structure component is contained by 0.01% by weight or less to the main component and preferably by 0.005% by weight or less. It goes without saying that the content of the impurity contained ketone structure component is preferred to be [0% by weight]. However, it is the best to be less than the detection limit of a detector.

When the content of the impurity contained ketone structure component is determined, a method below is preferably used. For instance, a gas chromatography unit with a mass analyzer as a detector and a hydrogen flame ionization detector can be used to analyze. When a gas chromatography unit with a mass analyzer as a detector is used, a peak of a particular impurity contained ketone structure component can be identified and at the same time a content thereof can be measured. However, since, in the points of measurement sensitivity and the dynamic range, the hydrogen flame ionization detector is superior to the mass analyzer, it is preferred to use the mass analyzer in the identification of a peak and the hydrogen flame ionization detector in quantitative determination. Since the hydrogen flame ionization detector cannot detect water in principle, a combinatorial use thereof is particularly preferred. It is practical to take in outputs from the detector in a computer to use values of areas of the respective peaks to obtain the contents and thereby a time and cost necessary for a determination process can be largely reduced.

Examples of the main components of the aliphatic ketone solvent include methyl ethyl ketone, methyl isobutyl ketone, cycloheptanone and cyclohexanone. The aliphatic ketone solvents are advantageous in that the solubility of organic compounds is high and halogen is not contained. The aliphatic ketone solvents are appropriate in the boiling point; accordingly, it can eliminate those problems that a coated layer is dried and solidified before sufficiently leveled after the coating to cause coating defects since the boiling point is too low; or, a temperature necessary for finishing the drying process becomes higher and a time for that becomes longer since the boiling point is too high. Among the aliphatic ketone solvents, cyclopentanone is particularly preferable from viewpoints of the solubility and time and temperature necessary for drying.

On the other hand, the impurity contained ketone structure component in the aliphatic ketone solvents includes components that have one or more, for instance, up to three more carbon atoms than the main component. These are as mentioned above readily generated as the impurity components and tend to adversely affect on the carrier transportability.

As an example, cyclopentanone that is particularly preferred will be described in detail. Examples of the impurity contained ketone structure component includes, for instance, at least one kind of cyclohexanone and 2-methyl-2-pentenone. Both substances can be detected by means of the mass analyzer and hydrogen flame ionization detector and can be readily identified from a fragmentation pattern measured by the mass analyzer.

In the next place, the organic compounds will be described. As the organic compounds, ones corresponding to an intended functional layer to be formed can be used and will be described in detail later. Furthermore, a content of the organic compound to the aliphatic ketone solvent is neither particularly restricted and can be appropriately selected corresponding to an intended functional layer to be formed and a coating method.

In the method of producing an organic electroluminescent device in the exemplary embodiment, it is preferred to measure in advance a content of an impurity contained ketone structure component in the aliphatic ketone solvent to determine whether the content is 0.01% by weight or less. The determination process is preferably carried out for every lot since normally a content of an impurity component may be different from lot to lot of an organic compound layer coating liquid.

Thus, when a process where a content of the impurity contained ketone structure component in the aliphatic ketone solvent is measured and whether the content reaches a predetermined value or not is determined (a determination method of an impurity content in an aliphatic ketone solvent for preparation of an organic electroluminescent device) is incorporated in a producing process, without actually preparing an organic compound layer coating liquid, producing an device and evaluating the performance thereof, whether the organic compound layer coating liquid can be used or not can be determined in advance. Accordingly, the yield can be improved and there is no necessity of discarding a coating liquid that cannot be used, resulting in largely contributing to the cost reduction at the production and a reduction of burden on environment.

Now, in the determination process, when the content of the impurity contained ketone structure component is measured and the content is determined to be the predetermined value or less, the aliphatic ketone solvent is used as it is in a later process (preparation and coating of an organic compound layer coating liquid). On the other hand, when the content is determined to exceed the predetermined value, the lot of the coating liquid is not used, or the solvent is purified by a distillation process, followed by once more measuring the content of the impurity component to determine whether the content reaches the predetermined value or not.

In what follows, a constitution and a method of producing an organic electroluminescent device in the exemplary embodiment will be described in detail with reference to the drawings.

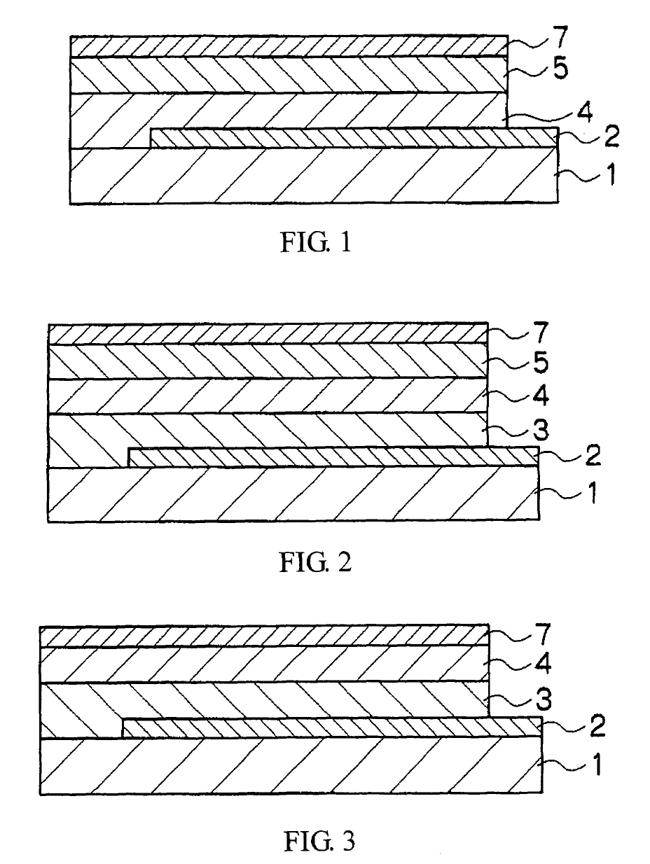

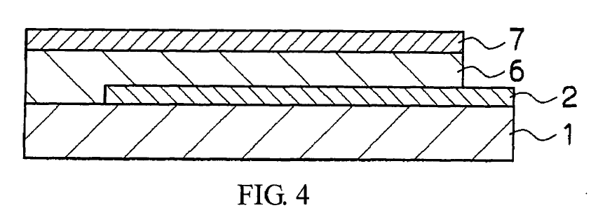

FIGS. 1 through 4 are schematic sectional views for explaining a layer structure of an organic electroluminescent device of the invention, FIGS. 1 through 3 being an example where a plurality of organic compound layers is formed, and FIG. 4 being an example where one organic compound layer is formed. In FIGS. 1 through 4, ones having a similar function are explained with same reference numerals.

An organic electroluminescent device shown in FIG. 1 is formed by, on a transparent insulating substrate 1, sequentially layering a transparent electrode 2, a luminescent layer 4, an electron transport layer 5 and a rear electrode 7. An organic electroluminescent device shown in FIG. 2 is formed by, on a transparent insulating substrate 1, sequentially layering a transparent electrode 2, a hole transport layer 3, a luminescent layer 4, an electron transport layer 5 and a rear electrode 7. An organic electroluminescent device shown in FIG. 3 is formed by, on a transparent insulating substrate 1, sequentially layering a transparent electrode 2, a hole transport layer 3, a luminescent layer 4 and a rear electrode 7. An organic electroluminescent device shown in FIG. 4 is formed by, on a transparent insulating substrate 1, sequentially layering a transparent electrode 2, a luminescent layer 6 having the carrier transportability and a rear electrode 7. Other than those layers described above, a hole injection layer or an electron injection layer may be disposed as needs arise.

In order to take out luminescence, the transparent insulating substrate 1 is preferably transparent one. Substrates such as glass or a plastic film can be used. Furthermore, the transparent electrode 2, similarly to the transparent insulating substrate, in order to take out the luminescence, is preferably transparent, and, in order to inject holes, is preferably large in the work function. Films of oxides such as indium tin oxide (ITO), tin oxide (NESA), indium oxide and zinc oxide or deposited or sputtered gold, platinum and palladium can be preferably used.

In the electron transport layer 5, a charge transporting material is used. Examples of the charge transporting materials include pyridinoquinolino complex of aluminum or beryllium, oxadiazole derivative, nitro-substituted fluorenone derivative, diphenoquinone derivative, thiopyran dioxide and fluorenylidene methane derivative. These are normally disposed by means of a vapor deposition method. Still furthermore, as the charge transporting material, polymers such as polyphenylene vinylenes and polyfluorenes can be cited. Such a polymer may be applied by a wet coating method (the organic compound coating). However, it is preferable that a solvent does not dissolve an already disposed undercoat layer.

When the electron injection layer is disposed between the electron transport layer 5 and the rear electrode 7 in order to improve the electron injectability from a negative electrode, one that has a function of injecting electrons from a negative electrode can be used as a material. That is, materials similar to the electron transport materials can be used.

In the hole transport layer 3, a hole transporting material is used. Examples of such hole transporting materials include polymers containing a ternary aromatic amine skeleton, carbazole skeleton, stilbene skeleton or arylhydrazone skeleton as a repeating unit in a main chain or polymers containing the above skeleton as a pendant in a polymer main chain. Such polymers may be applied by a wet coating method (the organic compound coating). However, it is preferable that a solvent does not dissolve an already disposed undercoat layer.

When the hole injection layer is formed between the transparent electrode 2 and the hole transport layer 3 in order to improve the hole injectability from a positive electrode, one that has a function of injecting holes from a positive electrode can be used as a material. As such a material, a vapor deposition layer of copper phthalocyanine can be used. However, a mixture (common name: PEDOT) of polystyrene sulfonic acid and poly(2,3-dioxyethynilthiophene) dispersed in an aqueous solvent can be more preferably used, since it has very low solubility in an aliphatic ketone solvent.

A luminescent material is used in the luminescent layer 4. Examples of the luminescent materials include, for instance, pyridinoquinolino complexes of a metal such as aluminum or beryllium. These materials may be disposed by a vapor deposition method. Examples of the luminescent materials include, luminescent polymers such as polyphenylene vinylenes and polyfluorenes. Such polymers may be applied by a wet coating method (the organic compound coating). However, it is preferable that a solvent does not dissolve an undercoat layer that is already disposed.

In the rear electrode 7, a metal that can be vacuum deposited and is small in the work function for injecting electrons is used. Magnesium, aluminum, silver, indium and alloys thereof are particularly preferable. Furthermore, a protective layer may be further disposed on the rear electrode 7 to inhibit the device from deteriorating due to moisture or oxygen.

Examples of specific materials of the protective layer include metals such as In, Sn, Pb, Au, Cu, Ag and Al, metal oxides such as MgO, SiO₂ and TiO₂ and resins such as a polyethylene resin, polyurea resin and polyimide resin. When the protective layer is formed, a vacuum deposition method, sputtering method, plasma polymerization method, CVD method or coating method can be used.

Among the respective layers of an organic electroluminescent device having the above-mentioned respective layer structures, when a layer is disposed by use of a wet coating method (the organic compound layer coating), generally, a spin coating method, dip coat method or inkjet method is used to layer.

Film thicknesses of the hole transport layer 3, luminescent layer 4 and electron transport layer 5 to be formed are respectively preferably 0.1 μm or less and more preferably in the range of 0.03 to 0.08 μm. Furthermore, a film thickness of the luminescent layer 6 with the carrier transportability is preferably in the range of substantially 0.03 to 0.2 μm. Still furthermore, film thicknesses when the hole injection layer and electron injection layer are formed, respectively, are preferably substantially equal to or thinner than that of the hole transport layer 3 and electron transport layer 5.

Furthermore, the organic electroluminescent device of the exemplary embodiment in can be sufficiently emitted when between a pair of electrodes for instance a voltage of 4 to 20 V and a DC current having the current density in the range of 1 to 200 mA/cm² are applied.

### EXAMPLES

In what follows, the embodiment will be further specifically described with reference to examples. However, the embodiment is not restricted to the respective examples.

Firstly, an aliphatic ketone solvent that is used to prepare an organic electroluminescent device will be described. As the aliphatic ketone solvent, five kinds of cyclopentanones below are used. The purities of the respective cyclopentanones and contents (a sum total of two kinds) of impurity contained ketone structure component (cyclohexanone and 2-methyl-2-pentenone, respectively, having one and two more carbon atoms than cyclopentanone as a main component) thereof are shown below. The content is a value obtained from a peak area obtained by measuring with a gas chromatography system (trade name: GC-17A, equipped with FID detector, produced by Shimadzu Corporation). In what follows, the content is obtained similarly.

TABLE 1

| | | Content of cyclohexanone and |
|---|---|---|
| General Designation | Purity | 2-methyl-2-pentenone |
| CPN-A (obtained by distilling | 99.98% | 0.006% |
| CPN-D under reduced pressure) | | |
| CPN-B (obtained by distilling | 99.98% | 0.010% |
| CPN-E under reduced pressure) | | |
| CPN-C (commercially obtained) | 99.96% | Incapable of detecting (0%) |
| CPN-D (commercially obtained) | 99.93% | 0.025% |
| CPN-E (commercially obtained) | 99.97% | 0.014% |

Cyclohexanones that are used as the aliphatic ketone solvent are two kinds below. The purities and contents of impurity contained ketone structure component (cycloheptanone having one more carbon atom than cyclohexanone as a main component) of the respective cyclohexanones are shown below.

TABLE 2

| General Designation | Purity | Content of cycloheptanone |
|---|---|---|
| CHN-A (obtained by distilling | 99.99% | 0.006% |
| CPN-B under reduced pressure) | | |
| CHN-B (commercially obtained) | 99.98% | 0.018% |

Methyl ethyl ketones that are used as the aliphatic ketone solvent are two kinds below. The purities and contents of impurity contained ketone structure component (diethyl ketone and methyl propyl ketone, respectively, having one and two more carbon atoms than methyl ethyl ketone as a main component) of the respective methyl ethyl ketones are shown below.

TABLE 3

| | | Content of diethyl ketone |
|---|---|---|
| General Designation | Purity | and methyl propyl ketone |
| MEK-A (obtained by distilling | 99.97% | 0.009% |
| MEK-B under reduced pressure) | | |
| MEK-B (commercially obtained) | 99.97% | 0.026% |

Methyl isobutyl ketones that are used as the aliphatic ketone solvent are two kinds below. The purities and contents of impurity contained ketone structure component (ethyl isobutyl ketone and normal propyl isobutyl ketone having one more carbon atom than methyl isobutyl ketone as a main component) of the respective methyl isobutyl ketones are shown below.

TABLE 4

| | | Content of ethyl isobutyl |
|---|---|---|
| | | ketone and normal propyl |
| General Designation | Purity | isobutyl ketone |
| MIBK-A (obtained by distilling | 99.98% | Incapable of |
| MIBK-B under reduced pressure) | | detecting (0%) |
| MIBK-B (commercially obtained) | 99.93% | 0.036% |

### Example 1

A CPN-A solution of 5% by mass of charge transporting polyester having a repeating structure (I-1) below (a weight average molecular weight based on styrene: substantially 120,000) is prepared, followed by filtering with a 0.1 μm polytetrafluoroethylene (PTFE) filter. The solution is coated, by means of a spin coating method, on a glass substrate on which a slit ITO electrode having a width of 2 mm is formed by etching to form a charge transport layer having a film thickness of substantially 0.1 μm. The coated glass substrate is left until it can be confirmed that a formed film does not have the fluidity and can be transported to a next step without problems, followed by forming an electron transport layer having a thickness of 0.05 μm from a compound (I-2) illustrated below by use of the vacuum deposition method. Finally, a Mg—Ag alloy is codeposited to form a rear electrode having a width of 2 mm and a thickness of 0.15 μm so as to intersect with the ITO electrode. An effective area of a prepared organic electroluminescent device is 0.04 cm².

With thus prepared organic electroluminescent device, with an ITO electrode side as a plus electrode and the Mg—Ag rear electrode as a minus electrode in a vacuum (1.33×10⁻¹ Pa), the brightness [cd/m²] under application of 5 V and the brightness-current efficiency at the brightness of 1000 cd/m² are measured. Results are shown in Table 5.

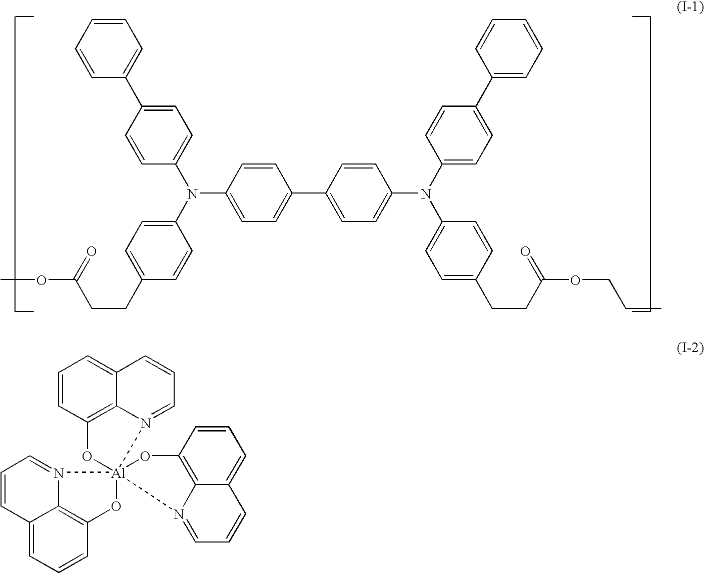

### Example 2

An organic electroluminescent device is prepared and evaluated similarly to example 1 except that CPN-B is used in place of CPN-A. Results are shown in Table 5.

### Example 3

An organic electroluminescent device is prepared and evaluated similarly to example 1 except that CPN-C is used in place of CPN-A. Results are shown in Table 5.

### Comparative Example 1

An organic electroluminescent device is prepared and evaluated similarly to example 1 except that CPN-D is used in place of CPN-A. Results are shown in Table 5.

### Comparative Example 2

An organic electroluminescent device is prepared and evaluated similarly to example 1, except that CPN-E is used in place of CPN-A. Results are shown in Table 5.

TABLE 5

| | | | Brightness-current |
|---|---|---|---|
| | Solvent | Brightness [cd/m²] | Efficiency at Brightness |
| | Used | Under 5 V Application | of 1000 cd/m² [cd/A] |
| Example 1 | CPN-A | 1256 | 6.0 |
| Example 2 | CPN-B | 1217 | 6.3 |
| Example 3 | CPN-C | 1323 | 6.0 |
| Comparative | CPN-D | 661 | 2.8 |
| Example 1 | | | |
| Comparative | CPN-E | 1058 | 4.5 |
| Example 2 | | | |

### Example 4

A CPN-A solution of 5% by mass of charge transporting polyester having a repeating structure (I-3) below (a weight average molecular weight based on styrene: about 80,000) is prepared, followed by filtering with a 0.1 μm polytetrafluoroethylene (PTFE) filter. The solution is coated, by means of a spin coating method, on a glass substrate on which a slit ITO electrode having a width of 2 mm is formed by etching to form a charge transport layer having a film thickness of about 0.1 μm. The coated glass substrate is left until it can be confirmed that a formed film does not have the fluidity and can be transported to a next step without problems, followed by forming an electron transport layer having a thickness of 0.05 μm from a compound (1-2) illustrated below by use of the vacuum deposition method. Finally, a Mg—Ag alloy is codeposited to form a rear electrode having a width of 2 mm and a thickness of 0.15 μm so as to intersect with the ITO electrode. An effective area of a prepared organic electroluminescent device is 0.04 cm².

With thus prepared organic electroluminescent device, with an ITO electrode side as a plus electrode and the Mg—Ag rear electrode as a minus electrode in a vacuum (1.33×10⁻¹ Pa), the brightness [cd/m²] under application of 5 V and the brightness-current efficiency [cd/A] at the brightness of 1000 cd/m² are measured. Results are shown in Table 6.

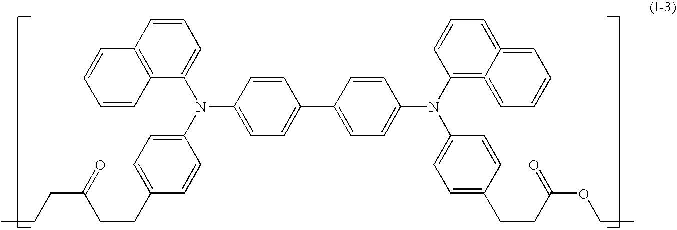

### Example 5

An organic electroluminescent device is prepared and evaluated similarly to example 4 except that CPN-B is used in place of CPN-A. Results are shown in Table 6.

### Example 6

An organic electroluminescent device is prepared and evaluated similarly to example 4, except that CPN-C is used in place of CPN-A. Results are shown in Table 6.

### Comparative Example 3

An organic electroluminescent device is prepared and evaluated similarly to example 4 except that CPN-D is used in place of CPN-A. Results are shown in Table 6.

### Comparative Example 4

An organic electroluminescent device is prepared and evaluated similarly to example 4, except that CPN-E is used in place of CPN-A. Results are shown in Table 6.

TABLE 6

| | | Brightness Under | Brightness-current |
|---|---|---|---|
| | Solvent | Application of 5 V | Efficiency at the Brightness |
| | Used | [cd/m²] | of 1000 cd/m² [cd/A] |
| Example 4 | CPN-A | 1451 | 7.4 |
| Example 5 | CPN-B | 1452 | 7.8 |
| Example 6 | CPN-C | 1512 | 7.3 |
| Comparative | CPN-D | 907 | 5.1 |
| Example 3 | | | |
| Comparative | CPN-E | 1149 | 7.2 |
| Example 4 | | | |

### Example 7

A CPN-A solution of 5% by mass of charge transporting polyurethane having a repeating structure (I-4) below (a weight average molecular weight based on styrene: about 120,000) is prepared, followed by filtering with a 0.1 μm polytetrafluoroethylene (PTFE) filter. The solution is coated, by means of a spin coating method, on a glass substrate on which a slit ITO electrode having a width of 2 mm is formed by etching to form a charge transport layer having a film thickness of substantially 0.1 μm. The coated glass substrate is left until it can be confirmed that a formed film does not have the fluidity and can be transported to a next step without problems, followed by coating a cyclohexanone solution of 5% by mass of π conjugate polymer having a repeating structure (I-5) below (weight average molecular weight based on styrene: about 65,000) after filtering with a 0.1 μm polytetrafluoroethylene (PTFE) filter on the charge transport layer as a luminescent material to form a luminescent layer having a thickness of about 0.1 μm, finally followed by codepositing a Mg—Ag alloy to form a rear electrode having a width of 2 mm and a thickness of 0.15 μm so as to intersect with the ITO electrode. An effective area of a prepared organic electroluminescent device is 0.04 cm².

With thus prepared organic electroluminescent device, with an ITO electrode side as a plus electrode and the Mg—Ag rear electrode as a minus electrode in a vacuum (1.33×10⁻¹ Pa), the brightness [cd/m²] under application of 5 V and the brightness-current efficiency [cd/A] at the brightness of 1000 cd/m² are measured. Results are shown in Table 7.

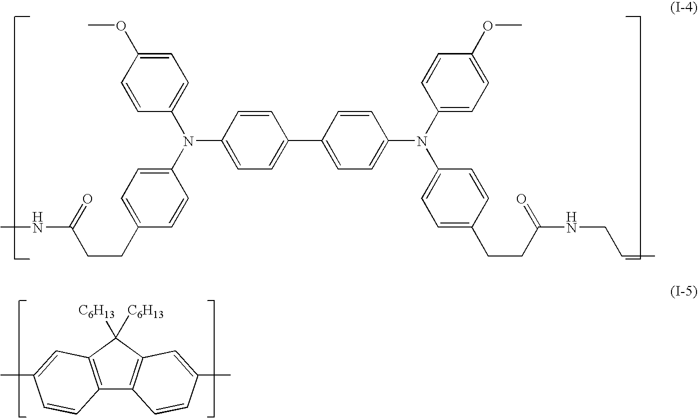

### Example 8

An organic electroluminescent device is prepared and evaluated similarly to example 7, except that CPN-B is used in place of CPN-A. Results are shown in Table 7.

### Example 9

An organic electroluminescent device is prepared and evaluated similarly to example 7, except that CPN-C is used in place of CPN-A. Results are shown in Table 7.

### Comparative Example 5

An organic electroluminescent device is prepared and evaluated similarly to example 7, except that CPN-D is used in place of CPN-A. Results are shown in Table 7.

### Comparative Example 6

An organic electroluminescent device is prepared and evaluated similarly to example 7, except that CPN-E is used in place of CPN-A. Results are shown in Table 7.

TABLE 7

| | | Brightness Under | Brightness-current |
|---|---|---|---|
| | Solvent | Application of 5 V | Efficiency at Brightness |
| | Used | [cd/m²] | of 1000 cd/m² [cd/A] |
| Example 7 | CPN-A | 928 | 3.2 |
| Example 8 | CPN-B | 908 | 3.1 |
| Example 9 | CPN-C | 987 | 3.1 |
| Comparative | CPN-D | 345 | 0.96 |
| Example 5 | | | |
| Comparative | CPN-E | 947 | 2.9 |
| Example 6 | | | |

### Example 10

On a glass substrate on which a 2 mm wide slit ITO electrode is formed by etching, Baytron (mixed aqueous dispersion of polymer of polyethylene dioxide thiophene and polystyrene sulfonic acid, produced by Bayer Corp.) is coated by means of a spin coating method, heated and dried to form a hole injection layer having a film thickness of 0.1 μm. Thereon, a solution obtained by preparing a toluene solution of 5% by mass of charge transporting polyether (weight average molecular weight based on polystyrene: about 85,000) having a repeating structure (I-6) below, followed by filtering with a 0.1 μm polytetrafluoroethylene (PTFE) filter is coated by means of a spin coating method to form a charge transport layer having a film thickness of about 0.1 μm.

The coated glass substrate is left until it can be confirmed that a formed film does not have the fluidity and can be transported to a next step without problems, followed by coating a solution obtained by filtering with a 0.1 μm polytetrafluoroethylene (PTFE) filter a CHN-A solution of 5% by mass of π conjugate polymer having a repeating structure (I-7) below (weight average molecular weight based on styrene: about 49,000) as a luminescent material on the charge transport layer to form a luminescent layer having a thickness of about 0.1 μm, finally followed by codepositing a Mg—Ag alloy to form a rear electrode having a width of 2 mm and a thickness of 0.15 μm so as to intersect with the ITO electrode. An effective area of a prepared organic electroluminescent device is 0.04 cm².

With thus prepared organic electroluminescent device, with an ITO electrode side as a plus electrode and the Mg—Ag rear electrode as a minus electrode in a vacuum (1.33×10⁻¹ Pa), the brightness [cd/m²] under application of 5 V and the brightness-current efficiency [cd/A] at the brightness of 1000 cd/m² are measured. Results are shown in Table 8.

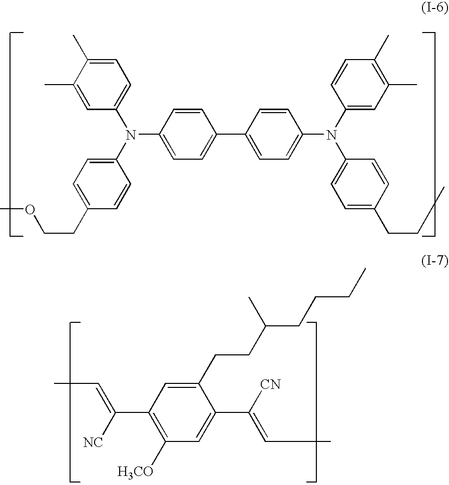

### Example 11

An organic electroluminescent device is prepared and evaluated similarly to example 10 except that CPN-B is used in place of CPN-A. Results are shown in Table 8.

### Example 12

An organic electroluminescent device is prepared and evaluated similarly to example 10, except that CPN-C is used in place of CPN-A. Results are shown in Table 8.

### Comparative Example 7

An organic electroluminescent device is prepared and evaluated similarly to example 10, except that CPN-D is used in place of CPN-A. Results are shown in Table 8.

### Comparative Example 8

An organic electroluminescent device is prepared and evaluated similarly to example 10 except that CPN-E is used in place of CPN-A. Results are shown in Table 8.

TABLE 8

| | | Brightness Under | Brightness-current |
|---|---|---|---|
| | Solvent | Application of 5 V | Efficiency at Brightness |
| | Used | [cd/m²] | of 1000 cd/m² [cd/A] |
| Example 10 | CPN-A | 1908 | 5.1 |
| Example 11 | CPN-B | 1987 | 5.3 |
| Example 12 | CPN-C | 1967 | 5.1 |
| Comparative | CPN-D | 795 | 2.9 |
| Example 7 | | | |
| Comparative | CPN-E | 1689 | 4.0 |
| Example 8 | | | |

### Example 13

An organic electroluminescent device is prepared and evaluated similarly to example 1 except that CHN-A is used in place of CPN-A. Results are shown in Table 9.

### Comparative Example 9

An organic electroluminescent device is prepared and evaluated similarly to example 1, except that CHN-B is used in place of CPN-A. Results are shown in Table 9.

TABLE 9

| | | Brightness Under | Brightness-current |
|---|---|---|---|
| | Solvent | Application of 5 V | Efficiency at Brightness |
| | Used | [cd/m²] | of 1000 cd/m² [cd/A] |
| Example 13 | CHN-A | 1143 | 5.9 |
| Comparative | CHN-B | 435 | 1.8 |
| Example 9 | | | |

### Example 14

An organic electroluminescent device is prepared and evaluated similarly to example 4, except that MEK-A is used in place of CPN-A. Results are shown in Table 10.

### Comparative Example 10

An organic electroluminescent device is prepared and evaluated similarly to example 1, except that MEK-B is used in place of CPN-A. Results are shown in Table 10.

TABLE 10

| | | Brightness Under | Brightness-current |
|---|---|---|---|
| | Solvent | Application of 5 V | Efficiency at Brightness |
| | Used | [cd/m²] | of 1000 cd/m² [cd/A] |
| Example 14 | MEK-A | 1483 | 7.9 |
| Comparative | MEK-B | 328 | 3.6 |
| Example 10 | | | |

### Example 15

An organic electroluminescent device is prepared and evaluated similarly to example 7 except that MIBK-A is used in place of CPN-A. Results are shown in Table 11.

### Comparative Example 11

An organic electroluminescent device is prepared and evaluated similarly to example 7 except that MIBK-B is used in place of CPN-A. Results are shown in Table 11.

TABLE 11

| | | Brightness Under | Brightness-current |
|---|---|---|---|
| | Solvent | Application of 5 V | Efficiency at Brightness |
| | Used | [cd/m²] | of 1000 cd/m² [cd/A] |
| Example 15 | MIBK-A | 888 | 2.6 |
| Comparative | MIBK-B | 45 | 0.16 |
| Example 11 | | | |

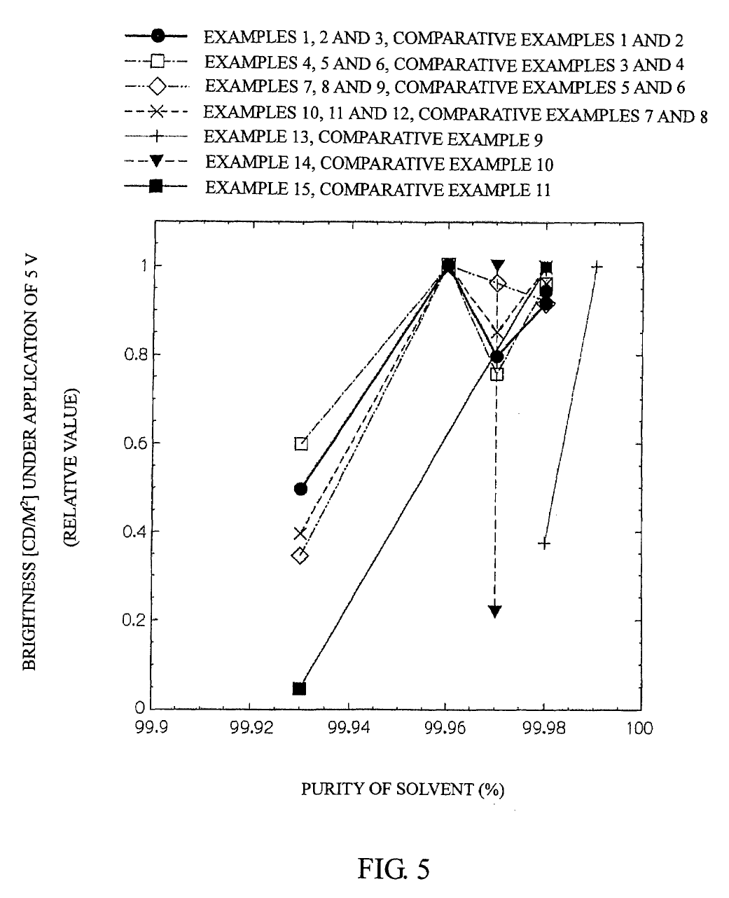

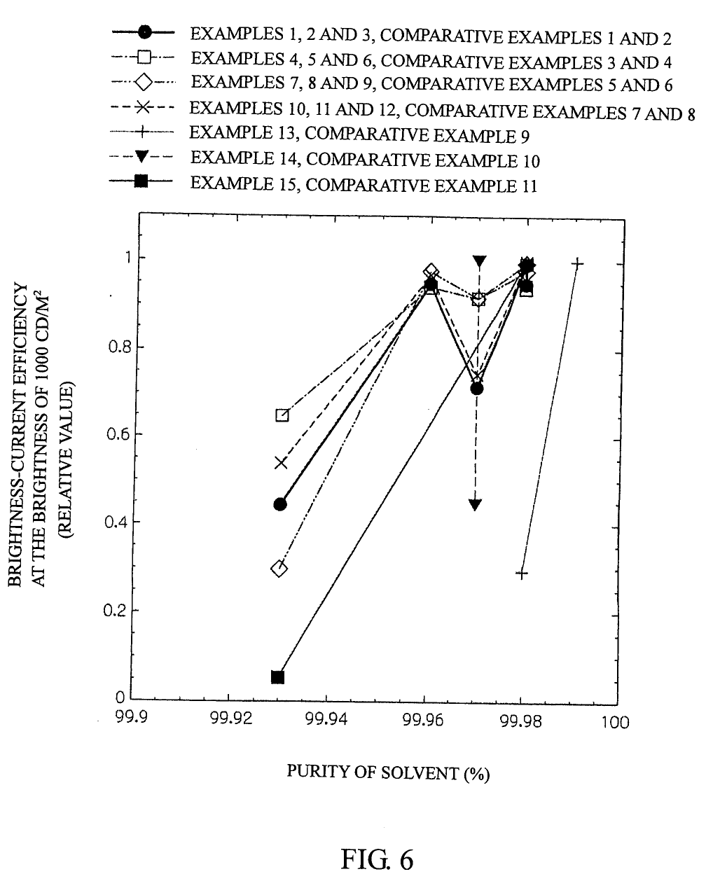

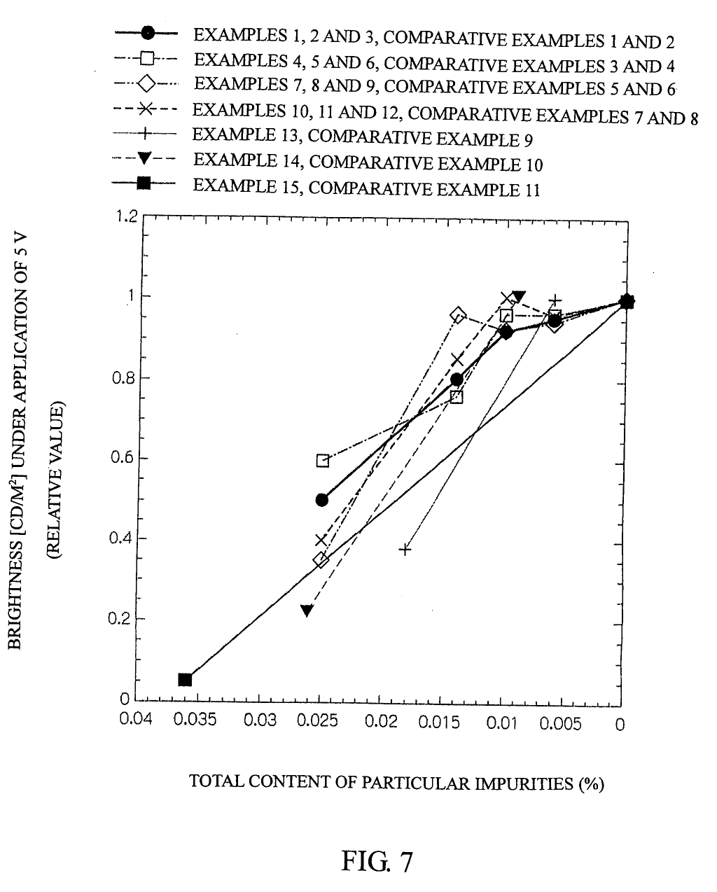

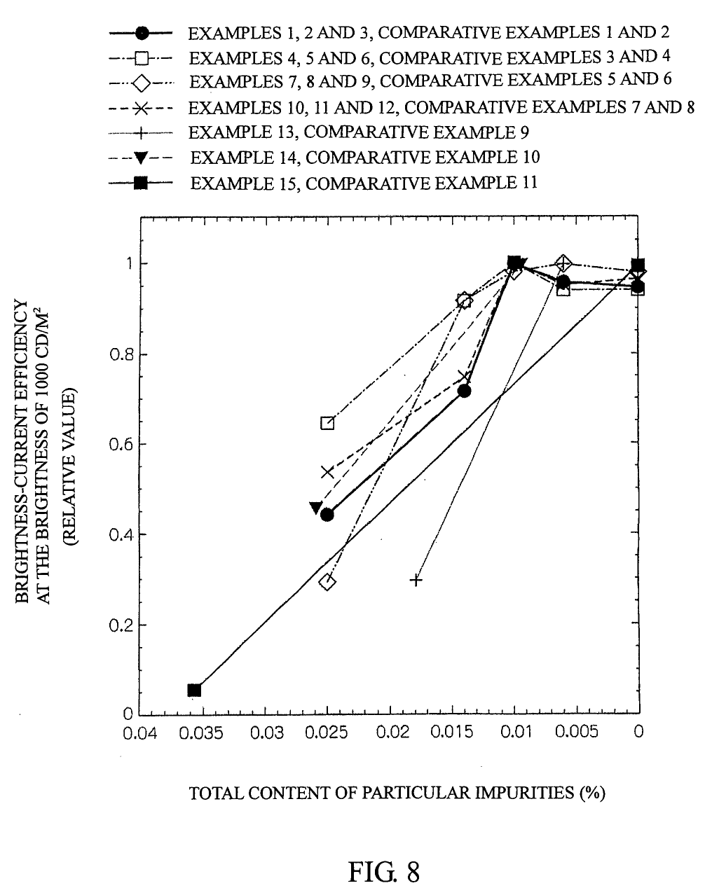

Numerical values obtained in the respective examples and comparative examples cited in Tables 5 through 11 are normalized to the maximum values in the respective tables and the normalized values are shown as graphs in FIGS. 5 through 8. From FIGS. 5 and 6 where the purity of the solvent is shown on a horizontal axis, it is found that there is rough tendency that more excellent performance can be obtained when a higher purity solvent is used, however, it is not possible to specify the purity that is a criterion for producing an device having stable luminescent performance. On the other hand, in FIGS. 7 and 8 where a total content of particular impurity components is shown on a horizontal axis, the fluctuation between 7 graph lines is small and shows an increasing tendency toward a right side in the graph; accordingly, it is obvious that a criterion for producing devices having stable luminescent performance can be determined.

With cyclopentanone used as the aliphatic ketone solvent as an example, a more detailed description will be given. When commercially available CPN-D having the purity of 99.93% and commercially available CPN-E having the purity of 99.97% are used, initial performances tend to be low and the fluctuations due to difference of the device configuration are large. In order to improve the performances thereof, it is necessary to distill to improve the purity (CPN-A: 99.93% →99.98%, CPN-B: 99.94% →99.98%). However, when the commercially available product CPN-C (purity: 99.96%) is used as it is, the initial performance is more excellent and the fluctuation due to the device configuration is less than when the distilled ones are used. Accordingly, it is found that the purity of cyclopentanone is not a unique index for determining the applicability to device production.

On the other hand, in FIGS. 7 and 8 where a total content of particular impurities, that is, cyclohexanone and 2-methyl-2-pentenone is shown on a horizontal axis, it is found that, in the initial performance and the fluctuation due to the difference of device configuration, the smaller the total content of two components becomes, the more excellent the results are. In more detail, when the total content is 0.014%, depending on the device configurations, excellent performances can be obtained; however, the fluctuations are large. When the total content is 0.01%, the fluctuations become obviously small. Accordingly, the total content of the two components is, though most preferably not to be detected, preferably 0.01% or less and depending on the device configuration even when the content is 0.014% or less excellent initial characteristics can be obtained.

That is, it is found that when a method of producing the invention of organic electroluminescent devices is applied, by use of an aliphatic ketone solvent that is a halogen-free solvent that is less in the environmental burden, devices excellent in the brightness and brightness-luminescence efficiency can be assuredly obtained.

Furthermore, by neither carrying out device preparation/evaluation nor applying an operation that necessitates a large amount of energy such as distillation, a purchased solvent can be determined whether it can be used as it is to produce devices or not. Accordingly, it becomes unnecessary to discard defective coating liquids, producing costs can be reduced and environmental burden can be largely reduced.

All publications, patent applications, and technical standards mentioned in this specification are herein incorporated by reference to the same extent as if such individual publication, patent application, or technical standard was specifically and individually indicated to be incorporated by reference.

It will be obvious to those having skill in the art that many changes may be made in the above-described details of the exemplary embodiments of the present invention. The scope of the invention, therefore, should be determined by the following claims.

What is claimed is:

1. A method for producing an organic electroluminescent device, comprising:
forming an organic compound layer with an organic compound layer coating liquid that contains an organic compound and an aliphatic ketone solvent,
the aliphatic ketone solvent containing about 0.01% by weight or less of an impurity contained ketone structure component.

2. The method for producing an organic electroluminescent device of claim 1, wherein a main component of the aliphatic ketone solvent is at least one selected from the group consisting of methyl ethyl ketone, methyl isobutyl ketone, cycloheptanone, cyclohexanone and cyclopentanone.

3. The method for producing an organic electroluminescent device of claim 1, wherein a main component of the aliphatic ketone solvent is cyclopentanone.

4. The method for producing an organic electroluminescent device of claim 1, wherein the number of carbon atoms of the impurity contained ketone structure component is one or more larger than that of a main component of the aliphatic ketone solvent.

5. The method for producing an organic electroluminescent device of claim 1, wherein the impurity contained ketone structure component is at least one of cyclohexanone or 2-methyl-2-pentenone.

6. The method for producing an organic electroluminescent device of claim 1, wherein, in advance, the content of the impurity contained ketone structure component in the aliphatic ketone solvent to be used is measured to determine whether or not the content is 0.01% by weight or less.

7. The method for producing an organic electroluminescent device of claim 6, wherein a main component of the aliphatic ketone solvent is cyclopentanone.

8. The method for producing an organic electroluminescent device of claim 6, wherein the number of carbon atoms of the impurity contained ketone structure component is one or more larger than that of a main component of the aliphatic ketone solvent.

9. The method for producing an organic electroluminescent device of claim 6, wherein the impurity contained ketone structure component is at least one of cyclohexanone or 2-methyl-2-pentenone.

10. An organic electroluminescent device produced by a method for producing an organic electroluminescent device, the method comprising:
forming an organic compound layer with an organic compound layer coating liquid that contains an organic compound and an aliphatic ketone solvent,
the aliphatic ketone solvent containing about 0.01% by weight or less of an impurity contained ketone structure component.

11. The method for producing an organic electroluminescent device of claim 10, wherein a main component of the aliphatic ketone solvent is cyclopentanone.

12. The method for producing an organic electroluminescent device of claim 10, wherein the impurity contained ketone structure component is at least one of cyclohexanone or 2-methyl-2-pentenone

13. The method for producing an organic electroluminescent device of claim 10, wherein, in advance, the content of the impurity contained ketone structure component in the aliphatic ketone solvent to be used is measured to determine whether or not the content is 0.01% by weight or less.

14. A method for determining an impurity content of an aliphatic ketone solvent for use in preparation of an organic electroluminescent device, comprising:
measuring a content of a impurity contained ketone structure component in an aliphatic ketone solvent to determine whether or not the content reaches a predetermined value.
